(19) United States
(12) Patent Application Publication
(10) Pub. No.: US 2010/0323446 A1
(43) Pub. Date: Dec. 23, 2010

(54) Method of collecting placental cells

(76) Inventors: Jill Renee Barnett, Denham Springs, LA (US); Edmund Burke, Jersey City, NJ (US); Melinda Long, Denham Springs, LA (US); Massi Malone, Baton Rouge, LA (US); Timothy J. Martinez, Denham Springs, LA (US); Pamela G. Shaver, Baton Rouge, LA (US); Rodney Vega, Donaldsonville, LA (US); Melissa Woodward, Saint Amant, LA (US)

Correspondence Address: JONES DAY, 222 E. 41ST. STREET, NEW YORK, NY, 10017, US

(21) Appl. No.: 12/793,620

(22) Filed: Jun. 3, 2010

Related U.S. Application Data
(60) Provisional application No. 61/184,732, filed on Jun. 5, 2009.

(51) Int. Cl. C12N 5/00

(52) U.S. Cl. 435/379

(57) ABSTRACT

Provided herein are improved methods of collecting and recovering placental cells from a mammalian placenta, comprising, e.g., perfusing a mammalian placenta in a closed system such as a sterile bag and folding the placenta at least once during perfusion. Such folding, and perfusion, can be performed mechanically.

This application claims benefit of U.S. Provisional Patent Application No. 61/184,732, filed Jun. 5, 2009, the disclosure of which is hereby incorporated by reference in its entirety.

### 1. FIELD

Provided herein are improved methods of collecting and recovering placental cells, e.g., total nucleated placental cells, placental perfusate cells, placental hematopoietic cells and/or multipotent placental cells, from a mammalian placenta by perfusion, e.g., by perfusion of a mammalian placenta within a sterile bag and/or with a particular method of physical manipulation of the placenta.

### 2. BACKGROUND

The placenta is an attractive source of therapeutic cells, e.g., placental multipotent cells, placental hematopoietic cells, and placental perfusate cells. See, e.g., Hariri, U.S. Pat. Nos. 7,045,148, 7,255,879 and 7,468,276; and U.S. Patent application Publication No. 2007/0275362 and U.S. patent application Ser. No. 12/240,956, filed Sep. 29, 2008, entitled “Tumor Suppression Using Placental Perfusate and Human Placenta-Derived Intermediate Natural Killer Cells,” the disclosures of each of which are incorporated by reference herein in their entireties. While placentas are readily available, it is desirable to maximize the number of cells obtained from each placenta by perfusion. There is thus a need for improved methods for the collection and recovery of placental cells from a post-partum mammalian placenta so as to recover increased numbers of cells from a single placenta.

### 3. SUMMARY

Provided herein are improved methods of collecting placental cells, e.g., total nucleated placental cells (also referred to herein as placenta derived perfusate cells (PDPCs), or, more specifically human placenta derived perfusate cells (HPDPCs), placental perfusate comprising total nucleated placental cells (PP), more specifically, human placental perfusate comprising total nucleated placental cells (HPP), and/or subsets of the placenta derived perfusate cells, e.g., tissue culture adherent placental cells, placental hematopoietic cells and/or multipotent placental cells, from a mammalian placenta, e.g., by perfusion of a post-partum placenta remaining after a successful birth.

Provided herein, in one aspect, is a method of collecting placental cells from a mammalian placenta comprising perfusing the mammalian placenta with a perfusion solution in an amount and for a time sufficient to collect a detectable amount of placental cells in said perfusion solution, wherein during said perfusion, said placenta is contained wholly within a sterile bag, e.g., a sealed sterile bag. The perfusion solution, passed through the placenta and containing placental cells, is referred to as perfusate, or, more specifically, human placental perfusate (HPP). “Collecting placental cells,” as used herein, encompasses the collection of HPP or HDPDCs, unless otherwise noted.

In a second aspect, provided herein is a method of collecting placental cells from a mammalian placenta comprising perfusing the mammalian placenta with a perfusion solution in an amount and for a time sufficient to collect a detectable amount of placental cells in said perfusion solution, wherein the placenta is folded once or a plurality of times during said perfusing. In a specific embodiment, during said perfusion, a first portion of the placenta is folded at least once towards a second portion of the placenta different from the first portion.

In a more specific embodiment, during folding of the placenta, the first portion of the placenta can be folded to one or more angles, e.g., any angle, relative to the second portion of the placenta. In various specific embodiments, the first portion of the placenta is folded towards the second portion of the placenta such that the first portion of the placenta is folded to an angle of between 0° and 180°, between 45° and 135°, between 55° and 125°, or between 75° and 105° relative to the second portion. In a specific embodiment, the first portion of the placenta is folded towards the second portion of the placenta such that the first portion of the placenta is disposed at an angle of about 90° relative to the second portion while the placenta is folded. In certain other embodiments, the first portion is folded to an angle of about 10, 20, 30, 40, 50, 60, 70, 80, 90, 100, 110, 120, 130, 140, 150, 160, 170 or 180 degrees relative to said second portion.

The surface area of the first portion of said placenta can be of any value greater than 5% and less than 100% of the total surface area of the placenta, when the placenta is laid out flat, umbilical cord side up. In a specific embodiment, the surface area of said first portion of said placenta is at least 5% of the surface area of said placenta. In a specific embodiment, the surface area of said first portion of said placenta is approximately equal to the surface area of said second portion of said placenta. In another specific embodiment, the surface area of the first portion of the placenta is approximately twice as much as the second portion of the placenta. In another specific embodiment, the surface area of the first portion of the placenta is approximately three times as much as the second portion of the placenta.

In another embodiment, the method of collecting placental cells from a mammalian placenta comprises perfusing said mammalian placenta with a perfusion solution in an amount and for a time sufficient to collect a detectable amount of said placental cells in said perfusion solution, wherein said placenta comprises a first plurality of portions and a second plurality of portions, and wherein at least one of said first plurality of portions is folded towards one of said second plurality of portions during said perfusing.

In a specific embodiment of the above methods, the placenta is folded once during perfusion. In another specific embodiment, the placenta is folded a plurality of times during perfusion.

In certain embodiments, the placenta is folded manually during perfusion. In a specific embodiment, said placenta is contained within a sterile bag, e.g., a sealed sterile bag, during said manual folding. In another specific embodiment, said placenta is not contained within a sterile bag, e.g., a sealed sterile bag, during said manual folding.

In certain other embodiments, the placenta is folded mechanically. In a specific embodiment, the placenta is folded mechanically, wherein said placenta is not contained within a sterile bag. In another specific embodiment, the placenta is folded mechanically by securing said bag system on a platform, wherein said platform comprises a first member (e.g., a plate) and a second member (e.g., a plate), and wherein said first member and second member are connected and rotatable such that said first member is aligned at an angle ranging from about 0° to about 180° to said second member. In a more specific embodiment, the first portion of said placenta is placed on said first member and said second portion of said placenta is placed on said second member of said placenta, and wherein said placenta is folded while said first member and second member are rotated.

In various embodiments, during mechanical folding, the first member is brought to an angle of between 0° and 180°, between 45° and 135°, between 55° and 125°, or between 75° and 105° relative to said second member. In a specific embodiment, the first member is folded to an angle of about 90° relative to said second member. In certain other embodiments, the first member is folded to an angle of about 10, 20, 30, 40, 50, 60, 70, 80, 90, 100, 110, 120, 130, 140, 150, 160, 170 or 180 degrees relative to said second member.

Where the placenta is folded more than once during perfusion, the folding can take place, e.g., at regular intervals, e.g., the time of folding is determined solely by elapsed time, or at irregular intervals, e.g., when the number of cells collected over a period of time drops below a predetermined threshold.

The perfusion in the methods provided herein can be performed for any period of time sufficient to collect a detectable amount of said placental cells, e.g., from approximately 2 minutes to approximately 48 hours. In various embodiments, the perfusion is maintained for two hours, for four hours, for 24 hours, for 24 hours, or for 48 hours after removal of said residual blood.

In a specific embodiment, said placenta has been drained of cord blood and flushed (e.g., perfused for a short time) to remove residual blood prior to said perfusing. In another specific embodiment, said perfusing is performed at at least four hours after removal of said residual blood. In another specific embodiment, said perfusing is performed at at least twelve hours after removal of said residual blood. In another specific embodiment, said perfusing is performed at at least 24 hours after removal of said residual blood. In another specific embodiment, said perfusing is performed using about 750 ml of said perfusion solution. In another specific embodiment, said perfusing is performed using a first volume of between about 30 ml and about 150 ml of said perfusion solution. In another specific embodiment, said placenta is perfused with between about 100 mL to 3000 mL of said perfusion solution In various other specific embodiments, the perfusion is performed using a first volume of about 100 ml to about 1000 mL, about 200 mL to about 900 mL, about 300 mL to about 800 mL, about 400 mL to about 800 mL, or about 750 mL of said perfusion solution. In specific embodiments, the perfusion is performed using a first volume of about 100 ml to about 1000 mL, about 200 mL to about 900 mL, about 300 mL to about 800 mL, about 400 mL to about 800 mL, or about 750 mL of said perfusion solution.

In another specific embodiment, said perfusing is continued using a second volume of about 30 ml to about 150 ml of said perfusion solution, said second volume being collected separately from said first volume. In other specific embodiments, the methods provided herein further comprise continuing the perfusion using a second volume of about first volume of about 100 ml to about 1000 mL, about 200 mL to about 900 mL, about 300 mL to about 800 mL, about 400 mL to about 800 mL, or about 750 mL of said perfusion solution, said second volume being collected separately from said first volume. In a specific embodiment, the methods provided herein further comprise continuing said perfusion using a second volume of about 750 ml of said perfusion solution, said second volume being collected separately from said first volume.

In another specific embodiment, said perfusing is performed for a plurality of times. In another specific embodiment, said perfusing is performed using a volume of about 30 ml to about 150 ml of said perfusion solution. In a more specific embodiment of the above methods, said perfusion solution comprises an anticoagulant. In another more specific embodiment, said perfusion solution comprises heparin, ethylene diamine tetra acetic acid (EDTA) or creatine phosphate dextrose (CPDA). In another more specific embodiment, said perfusion solution comprises a growth factor or a cytokine. In a more specific embodiment, said growth factor or cytokine is selected from the group consisting of a colony stimulating factor, interferon, erythropoietin, stem cell factor, thrombopoietin, an interleukin, granulocyte colony-stimulating factor, and any combination thereof.

In certain embodiments of the method, the placental cells are total nucleated placental cells, i.e., the nucleated cells from the placenta collected by perfusion of the placenta. In certain other embodiments of the method, the placental cells are CD34⁺ placental hematopoietic cells; tissue culture plastic-adherent CD34⁻ placental multipotent cells, or placental adherent cells (which may include adherent placental cells in addition to the tissue culture plastic-adherent CD34⁻ placental multipotent cells).

### 3.1 Definitions

The term “fold,” or “folding” as used herein refers to the bending or curving of a placenta so that one part of the placenta comes closer to another part.

The term “folded” as used herein refers to a state at which a placenta is being bent or forms an angular shape made by folding.

As used herein, the term “about,” when referring to a stated numeric value, indicates a value within plus or minus 10% of the stated numeric value.

As used herein, the term “SH2” refers to an antibody that binds an epitope on the cellular marker CD105. Thus, cells that are referred to as SH2⁺ are CD105⁺.

As used herein, the terms “SH3” and SH4” refer to antibodies that bind epitopes present on the cellular marker CD73. Thus, cells that are referred to as SH3⁺ and/or SH4⁺ are CD73^{±}.

A placenta has the genotype of the fetus that develops within it, but is also in close physical contact with maternal tissues during gestation. As such, as used herein, the term “fetal genotype” means the genotype of the fetus, e.g., the genotype of the fetus associated with the placenta from which particular isolated placental cells, as described herein, are obtained, as opposed to the genotype of the mother that carried the fetus. As used herein, the term “maternal genotype” means the genotype of the mother that carried the fetus, e.g., the fetus associated with the placenta from which particular isolated placental cells, as described herein, are obtained.

As used herein, the term “isolated cells” means cells that are substantially separated from other, different cells of the tissue, e.g., placenta, from which the cells are derived. Particular cells are “isolated” if at least 50%, 60%, 70%, 80%, 90%, 95%, or at least 99% of the cells with which the particular cells are naturally associated, or cells displaying a different marker profile, are removed from the particular cells, e.g., during collection and/or culture of the cells.

As used herein, “multipotent,” when referring to a cell, means that the cells has the ability to differentiate into some, but not all, types of cells of the body, or into cells having characteristics of some, but not all, types of cells of the body. In certain embodiments, for example, isolated placental cells that has the capacity to differentiate into cells having characteristics of neurogenic, osteogenic or adipogenic cells is a multipotent cell.

As used herein, the term “population of isolated cells” means a population of cells that is substantially separated from other cells of a tissue, e.g., placenta, from which the population of cells is derived.

As used herein, the term “total nucleated placental cells” means the nucleated cells from the placenta collected by perfusion of the placenta. The term refers to the types of cells obtained from the placenta by perfusion, and not to number. For example, the term allows for losses of cells during processing during and/or after perfusion.

As used herein, the term “placental multipotent cell” refers to tissue culture plastic-adherent cells obtained from a mammalian placenta, having multipotent cell characteristics, regardless of the number of passages after a primary culture. The term “placental multipotent cell” as used herein does not, however, refer to, and placental multipotent cells are not, however, trophoblasts, angioblasts, hemangioblasts, embryonic germ cells, embryonic stem cells, cells obtained from an inner cell mass of a blastocyst, or cells obtained from a gonadal ridge of a late embryo, e.g., embryonic germ cells. Cell are considered “multipotent cells” if the cells display attributes of multipotent cells, e.g., adherence to tissue culture plastic; a marker or gene expression profile associated with one or more types of multipotent cells; the ability to replicate at least 10-40 times, or more, in culture, and the ability to differentiate into cells displaying characteristics of differentiated cells of one or more of the three germ layers. The terms “placental multipotent cell” and “placenta-derived multipotent cell” may be used interchangeably. Unless otherwise noted herein, the term “placental” includes the umbilical cord. The isolated placental multipotent cells disclosed herein, in certain embodiments, differentiate in vitro under differentiating conditions, differentiate in vivo, or both.

As used herein, a placental cell is “positive” for a particular marker when that marker is detectable above background. For example, a placental cell is positive for, e.g., CD73 because CD73 is detectable on placental cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200⁺” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34⁻” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 is detectable using RT-PCR.

As used herein, “treat” encompasses the cure of, remediation of, improvement of, lessening of the severity of, or reduction in the time course of, a disease, disorder or condition, or any parameter or symptom thereof.

### 4. BRIEF DESCRIPTION OF THE FIGURES

FIG. 1 Side view of an example placental folding mechanism.

FIG. 2 Top view of an example placental folding mechanism.

### 5. DETAILED DESCRIPTION

### 5.1 Methods of Obtaining Placental Cells

Provided herein are improved methods of perfusing a mammalian, e.g., human, placenta to increase the number of placental cells collected compared to previously-described methods. Section 5.2 below described cell collection compositions suitable for perfusion of mammalian placenta. Section 5.3 below described methods for the collection and handling of placenta prior to perfusion. Section 5.4 below describes the improved method of perfusing mammalian placentas. Section 5.5 below describes the technique of collecting placental cells by perfusion wherein the placenta is enclosed in a sterile bag during perfusion.

### 5.2 Cell Collection Composition

Generally, placental cells can be obtained from a mammalian placenta by perfusion using a physiologically-acceptable solution, e.g., a cell collection composition. The passage of perfusion solution causes cells to disassociate from the placenta; the perfusion solution that is passed through the placenta is thus collected, thereby collecting placental cells in perfusate. Placental cells may subsequently be separated from the perfusate, and subsets of perfusate-derived cells may be separated from other subsets of perfusate-derived cells, e.g., as described in Section 5.5, below. A cell collection composition useful for the perfusion methods provided herein is described in detail in related U.S. Application Publication No. 20070190042, which is hereby incorporated by reference in its entirety.

The cell collection composition can comprise any physiologically-acceptable solution suitable for the collection and/or culture of cells, for example, a saline solution (e.g., phosphate-buffered saline, Kreb's solution, modified Kreb's solution, Eagle's solution, 0.9% NaCl. etc.), a culture medium (e.g., DMEM, HEPES-buffered DMEM (H.DMEM), Ham's F-12, etc.), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

### 5.3 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the placental cells harvested therefrom. For example, human placental cells collected by perfusion can be used, in light of the medical history, for personalized medicine for the infant associated with the placenta, or for parents, siblings or other relatives of the infant.

In certain embodiments, prior to recovery of placental cells, the umbilical cord blood and placental blood are removed prior to perfusion to collect placental cells. In certain other embodiments, prior to perfusion to collect placental cells, the umbilical cord blood and placental blood are removed.

In certain embodiments, after delivery, placental blood and umbilical cord blood is recovered for later use. In such embodiments, the placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. In certain embodiments, the placenta is gravity drained without further manipulation so as to minimize tissue disruption during cord blood recovery.

According to methods previously described, a placenta is typically transported from the delivery or birthing room to another location, e.g., a laboratory, for recovery of cord blood and collection of cells by perfusion. The placenta is preferably transported in a sterile, thermally insulated transport device, for example, by placing the placenta, with clamped proximal umbilical cord, in a sterile zip-lock plastic bag, which is then placed in an insulated container. In certain embodiments, the placenta is maintained at about room temperature (e.g., between about 20° C. to about 28° C.) during transport prior to perfusion. In another embodiment, the placenta is maintained at between about 1° C. to about 8° C. during transport prior to perfusion. In another embodiment, the placenta is transported in a cord blood collection kit substantially as described in pending U.S. Pat. No. 7,147,626, which is hereby incorporated by reference in its entirety. Preferably, the placenta is delivered to the laboratory four to twenty-four hours following delivery.

In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to perfusion, can be stored under sterile conditions and at either room temperature or at a temperature of about 1° C. to about 28° C. The placenta may be stored for a period of, e.g., four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta is preferably stored in an anticoagulant solution at a temperature of about 1° C. to about 25° C. (centigrade). Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental cells are collected.

Once the mammalian placenta is collected and prepared in accordance with the methods above, the placenta may be perfused to obtain placental cells.

### 5.4 Placental Perfusion

The improved method of perfusing mammalian placentas incorporates one, or both of two approaches: (1) perfusion of the placenta in a closed, sterile bag; and/or (2) folding of the placenta during perfusion.

5.4.1 General Aspects of Perfusion

Placental cells can be collected by perfusion, e.g., through the placental vasculature, using, e.g., a cell collection composition, as described in Section 5.2 above, as a perfusion solution. Perfusion can also be accomplished using a saline solution, e.g., phosphate buffered saline or 0.9% NaCl solution, or culture medium, as the perfusion solution. In one embodiment, a mammalian placenta is perfused by passage of perfusion solution through either or both of the umbilical artery and umbilical vein, e.g., by cannulation of the vessels. The umbilical vein can be, e.g., cannulated with a cannula, e.g., a TEFLON® or plastic cannula, that is connected to a sterile connection apparatus, such as sterile tubing. The flow of perfusion solution through the placenta may be accomplished passively, using, e.g., gravity flow into the placenta, or actively, e.g., using a pump. Preferably, the perfusion solution is forced through the placenta using a pump, e.g., a peristaltic pump. In one embodiment, the proximal umbilical cord is clamped during perfusion, and more preferably, is clamped within 4-5 cm (centimeter) of the cord's insertion into the placental disc.

In certain embodiments, cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 m mM beta-mercaptoethanol. The cells are washed after several minutes with a cold (e.g., 4° C.) cell collection composition.

Placental perfusion is typically performed in a sterile environment, for example, a clean laboratory, a clean room, or a sterile hood that is free from micro-organisms.

In preparation for perfusion, the placenta can be oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. In this method, cells are collected only from perfusate that has passed through the placental vasculature. Placental cells collected by this method, which can be referred to as a “closed circuit” method, are typically almost exclusively fetal (e.g., at least 90%, 95%, 98%, or 99% fetal).

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796, which is incorporated by reference herein in its entirety. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

The volume of perfusion solution used to collect placental cells may vary depending upon the number of cells to be collected, the size of the placenta, the number of collections to be made from a single placenta, etc. In various embodiments, the volume of perfusion solution may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion solution.

In certain embodiments, said perfusing is performed using about 750 ml of said perfusion solution. In certain other embodiments, said perfusing is performed using a first volume of between about 30 ml and about 150 ml of said perfusion solution. In another specific embodiment, said placenta is perfused with between about 100 mL to 3000 mL of said perfusion solution. In another specific embodiment, said placenta is perfused with between about 300 mL to 5000 mL In another specific embodiment, said placenta is perfused with between about 500 mL to 10,000 mL. In various other specific embodiments, the perfusion is performed using a first volume of about 100 ml to about 1000 mL, about 200 mL to about 900 mL, about 300 mL to about 800 mL, about 400 mL to about 800 mL, or about 750 mL of said perfusion solution. In specific embodiments, the perfusion is performed using a first volume of about 100 ml to about 1000 mL, about 200 mL to about 900 mL, about 300 mL to about 800 mL, about 400 mL to about 800 mL, or about 750 mL of said perfusion solution.

In another specific embodiment, said perfusing is continued using a second volume of about 30 ml to about 150 ml of said perfusion solution, said second volume being collected separately from said first volume. In other specific embodiments, the methods provided herein further comprise continuing the perfusion using a second volume of about 100 ml to about 1000 mL, about 200 mL to about 900 mL, about 300 mL to about 800 mL, about 400 mL to about 800 mL, or about 750 mL of said perfusion solution, said second volume being collected separately from said first volume. In other specific embodiments, the methods provided herein further comprise continuing the perfusion using a second volume of about 100 mL to 3000 mL, about 300 mL to 5000 mL, or about 500 mL to 10,000 mL of said perfusion solution, said second volume being collected separately from said first volume. In a specific embodiment, the methods provided herein further comprise continuing said perfusion using a second volume of about 750 ml of said perfusion solution, said second volume being collected separately from said first volume.

The placenta can be perfused a plurality of times over the course of several hours or several days. Where the placenta is to be perfused a plurality of times, it may be maintained or cultured under aseptic conditions in a container or other suitable vessel, and perfused with the cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a preferred embodiment, stem cells are collected at a time or times between about 8 hours and about 18 hours post-expulsion.

The placenta can be perfused using one of the volumes of perfusion solution, e.g., the volumes of perfusion solution described above, e.g., until the volume has been passed through the placenta and collected. The placenta may also be perfused using a particular volume of perfusion solution wherein the perfusion solution is recirculated through the placenta, e.g., through the placental vasculature, for a time, e.g., 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56 or 58 minutes, or for 1, 2, 3, 4, 5, 6, 7, 8, 9, or 10 hours, or more.

In a specific embodiment, said placenta has been drained of cord blood and flushed (e.g., perfused for a short time) to remove residual blood prior to said perfusing. In another specific embodiment, said perfusing is performed at at least four hours after removal of said residual blood. In another specific embodiment, said perfusing is performed at at least twelve hours after removal of said residual blood. In another specific embodiment, said perfusing is performed at at least 24 hours after removal of said residual blood. In another specific embodiment, said perfusing is performed using about 750 ml of said perfusion solution. In another specific embodiment, said perfusing is performed using a first volume of between about 30 ml and about 150 ml of said perfusion solution. In another specific embodiment, said perfusing is continued using a second volume of about 30 ml to about 150 ml of said perfusion solution, said second volume being collected separately from said first volume. In another specific embodiment, said perfusing is performed for a plurality of times. In another specific embodiment, said perfusing is performed for a plurality of times, wherein for each of said times, said perfusing is performed using a volume of about 30 ml to about 150 ml of said perfusion solution. In a more specific embodiment of the above methods, said perfusion solution comprises an anticoagulant. In another more specific embodiment, said perfusion solution comprises heparin, ethylene diamine tetra acetic acid (EDTA) or creatine phosphate dextrose (CPDA). In another more specific embodiment, said perfusion solution comprises a growth factor or a cytokine. In a more specific embodiment, said growth factor or cytokine is selected from the group consisting of a colony stimulating factor, interferon, erythropoietin, stem cell factor, thrombopoietin, an interleukin, granulocyte colony-stimulating factor, and any combination thereof.

5.4.2 Collection of Placental Cells Using a Sterile Bag

In one aspect of the perfusion method described herein, perfusion to collect placental cells, e.g., perfusion as described above, is performed in a closed system that is transportable. Such a system provides a far more flexible environment for collection of placental cells than, for example, perfusion in a clean room or under a sterile hood.

In certain embodiments, the closed system provided herein is a sterile bag. The sterile bag can be made, for instance, from a medically-acceptable plastic (e.g., polystyrene, polyvinyl chloride, polyester, polyolefin, polyurethane), other materials known in the art for use in handling clinical samples, or a combination of above materials. In certain embodiments, the sterile bag is made from materials suitable for heat sterilization, e.g., autoclaving, or sterilization by radiation (e.g., ultraviolet light, microwaves, e-beam, or the like). The sterile bag can be reusable or disposable, and is preferably transparent for visibility and tear-resistant. The sterility of the bag disclosed herein can be ensured, for example, by a sealing system provided on each bag, an airtight design, or by sterilization using methods known in the art, e.g., autoclave.

The material from which the bag is made can be relatively stiff or flexible. In certain embodiments, the plastic from which the bag is constructed is stiff enough to substantially support the weight of the placenta without folding when supported at the edges. In certain other embodiments, the plastic from which the bag is constructed is flexible enough to conform to the shape of the placenta when the placenta is inserted into the bag.

Typically, a mammalian, e.g., human placenta is disk-shaped and can be laid out substantially flat on a surface. Thus, in preferred embodiments, the bag is provided as a pouch, e.g., two flat sheets of materials sealed on, e.g., three sides, with an opening (e.g., an open side) suitable for receiving the placenta. The opening may be openable and resealable multiple times, or may be sealed permanently, e.g., using glue, adhesive, or heat sufficient to melt the plastic of the opening to itself.

In certain embodiments, the bag is provided as a standard size bag (e.g., “one size fits all”) suitable for receiving a mammalian placenta, e.g., a human placenta, flat without folding or spindling the placenta. The dimensions of the sterile bag provided herein are adaptable to the size of the placenta samples and the amount of perfusion solution which may be contained in the bag (see below). In certain embodiments, the bag can be square or rectangular, or can be substantially circular. The placenta may float freely within the bag, or the bag may be constructed so as to conform to the shape of the placenta. In certain embodiments, the bag is vacuum-sealed to the placenta; that is, the placenta is placed in the bag, the bag is sealed, and at least some, a majority, or all, of the air remaining in the bag is removed.

Because the bag is adapted to enable perfusion of a placenta, the bag, in certain embodiments, comprises one or more ports that allow the passage of perfusion solution, e.g., in one or more tubes, into the bag. In certain embodiments, the bag is adapted for pan method perfusion. In such an embodiment, for example, the bag comprises a port suitable for the passage of tubing into the bag, wherein the tubing ends are cannulated into the placental vessels, as described above. In this embodiment, perfusate comprising placental cells can be collected from the bag itself, e.g., by destructively or non-destructively opening the bag after completion of perfusion. Placental perfusate comprising placental cells may alternatively be collected from the tubing port, or from a second port designed to allow the evacuation of the perfusate from the bag.

In certain other embodiments, the sterile bag provided herein is adapted to the closed-circuit perfusion system. In this embodiment, the bag comprises a port suitable for the passage of tubing into the bag, wherein the tubing ends are cannulated into the placental vessels, as described above. Because the perfusate comprising placental cells is removed from the placenta via the tubing, the bag need not comprise an additional port for removal of perfusate. In embodiments in which the bag is to be used for both (e.g., simultaneous) pan method and closed circuit perfusion, the bag can be a bag adapted for pan method perfusion. The placing of plastic tubing is carried out with care, preferably under a sterile condition. An adhesive may be applied to the port to prevent leakage. Alternatively, the sterile bag can have pre-existing tubing or conduit means adapted for the closed-circuit perfusion system.

In using either of the bags described above, the tubing can be passed sterilely through the port(s) and out of the opening of the bag, for cannulation of the placenta, followed by placement of the placenta in the bag and sealing the opening. In another embodiment, the bag is provided to an end user with sterilized tubing already installed and passed through the ports, such that the bag is ready for use, e.g. once unpackaged.

Thus, in one embodiment, provided herein is a method of collecting placental cells by perfusion, comprising: (a) providing a closed sterile bag system; (b) admitting said placenta into said bag system; and (c) perfusing said placenta with a volume of perfusion solution for a time sufficient to collect a detectable amount of said cells in said perfusion solution. In a specific embodiment, said placenta has been drained of cord blood and flushed to remove residual blood prior to said perfusing. In another specific embodiment, said perfusing is performed by passing said perfusion solution into one or both of the umbilical artery and umbilical vein of said placenta.

5.4.3 Physical Manipulation of Placenta During Collection of Placental Cells

Placental cells can be collected from a mammalian placenta by physical manipulation during perfusion, wherein the physical manipulation can be, for instance, folding or bending a portion of placenta. Such folding or bending is performed, e.g., when the placenta is laid out flat on the surface of the placenta opposite the umbilical cord. The folding or bending can be performed manually, e.g., while an individual holds the placenta cupped in two hands, or can be performed mechanically.

In certain embodiments, collecting placental cells from a mammalian placenta comprises perfusing the mammalian placenta with a perfusion solution in an amount and for a time sufficient to collect a detectable amount of placental cells, wherein said placenta is folded once, or a plurality of times, during said perfusion. In one embodiment, during perfusion, a first portion of placenta is folded at least once towards a second portion of placenta that is different from the first portion. Preferably, the first portion comprises about half of the surface area of the placenta. The surface area of the first portion of said placenta can be of any value greater than 5% and less than 95% of the total surface area of the placenta, when the placenta is laid out flat, umbilical cord side up. In certain embodiments, the first portion comprises least about 5%, 10%, 15%, 20%, 25%, 30%, 35%, 40%, 45% or 50% of the surface area of the placenta, and the second portion comprises the remainder of the surface area of the placenta. In a specific embodiment, the surface area of the first portion is approximately equal to the surface area of the second portion. In another specific embodiment, the surface area of said first portion of said placenta is at least 5% of the surface area of said placenta. In another specific embodiment, the surface area of said first portion of said placenta is approximately equal to the surface area of said second portion of said placenta. In another specific embodiment, the surface area of the first portion of the placenta is approximately twice as much as the second portion of the placenta. In another specific embodiment, the surface area of the first portion of the placenta is approximately three times as much as the second portion of the placenta.

At the time the placenta is folded, the first portion of the placenta can be disposed at any angle relative to the second portion of the placenta. In certain embodiments, for example, the first portion is folded from between about 5° to about 175°, from between about 15° to about 165°, from between about 45° to about 135°, from between about 55° to about 125°, or from between about 75° to about 105° relative to the second portion. In certain embodiments, the first portion is folded towards the second portion such that the first portion and second portion come into contact with each other. In certain other embodiments, the first portion is folded towards the second portion such that the first portion and second portion do not come into contact with each other. In a specific embodiment, a first portion of placenta is folded at least once towards a second portion of placenta such that the first portion of placenta is disposed at an angle of about 45° to about 90° relative to the second portion while the placenta is folded. In certain other embodiments, the first portion is folded to an angle of about 10, 20, 30, 40, 50, 60, 70, 80, 90, 100, 110, 120, 130, 140, 150, 160, 170 or 180 degrees relative to said second portion.

During perfusion, the placenta can be folded once, or a plurality of times (that is, folded and unfolded a plurality of times), either simultaneously or sequentially. In certain embodiments, the placenta is folded, unfolded, and refolded continuously during perfusion. In certain other embodiments, the placenta is folded, unfolded, and refolded discontinuously during perfusion.

Where the placenta is folded more than once during perfusion, the folding can take place, e.g., at regular intervals, e.g., the time of folding can be determined solely by elapsed time. Folding during perfusion can, in certain embodiments, take place or be performed at irregular intervals, e.g., when the number of cells collected over a period of time drops below a predetermined threshold.

In certain embodiments, the placenta is contained within a sterile bag (see above) during folding. In such embodiments, the bag is generally folded along with the placenta. Tubes, passing through a port in the bag and carrying perfusion solution to and/or from the placenta, can be fixed to the bag in such a way as to avoid movement of the tubing through the port during folding and unfolding. Preferably, the portion of the tubing within the bag, e.g., between the port and the placenta, is slack enough to prevent accidental disconnection between the tubing and placenta during perfusion and folding/unfolding.

5.4.3.1 Folding of Placenta Mechanically

In certain embodiments, the folding of the placenta during perfusion is performed mechanically, e.g., by a mechanical device. Such a mechanical device can be any device that folds the placenta during perfusion according to the description above. The placenta can be placed on, or within, the device wherein the placenta is in a bag (e.g., a bag as described above), or can be placed on or within the device directly without use of a bag).

In certain embodiments, the device comprises one or more members (e.g., together constituting a platform) on which the placenta rests during folding. In a specific embodiment, the device comprises, e.g., a first member and a second member, that are rotatable with respect to each other so as to accomplish folding of a placenta. In a more specific embodiment, the first and second members are rectangular and of approximately equal size, and are attached to each other by a hinge along a common edge. In another specific embodiment, the device comprises a first member only, and the first member is deformable or foldable (e.g., in the same manner that a tortilla is deformable or foldable to facilitate formation of a taco shell) so as to accomplish folding of the placenta. Preferably, the device comprises a component that powers movement of the first member and second member relative to each other.

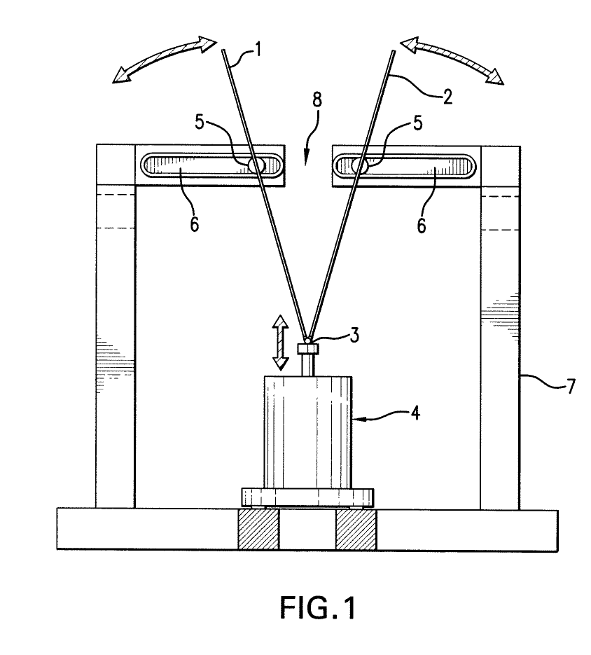

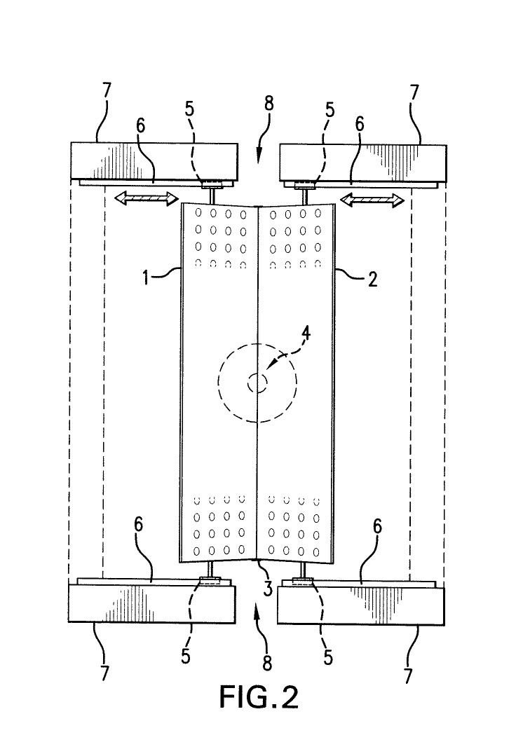

In one embodiment, an exemplary device is shown in FIGS. 1 and 2. The device comprises a platform comprising a first flat member 1 (e.g., a plate), and a second flat member 2, which are connected to each other through a hinge 3. The device further comprises a movable piston 4 connected to the platform at hinge 3, which provides driving force for movement of first member 1 and second member 2. First member 1 and second member 2 comprise wheels 5 on the side of the members adjacent to hinge 3. The wheels roll along wheel track 6 during movement of the members driven by piston 4. Wheel track 6 acts to support first member 1 and second member 2. The first member, second member, and piston are all supported by a frame 7.

During operation, piston 3 moves up and down, moving hinge 3, causing the portions of first member 1 and second member 2 attached to the hinge to move up and down, while the wheels 5 roll along wheel track 6, ultimately causing a folding motion of first member 1 and second member 2. In a specific embodiment, a gap 8 is present in frame 7 adjacent to the first and second members, e.g., to prevent pinching of plastic tubing used for collecting perfusate and placental cells. In another specific embodiment, no gap is present in frame 7.

When a placenta is placed on the platform, e.g., the first and second members, during perfusion, the device operates to fold the placenta during the perfusion process. In one embodiment, the placenta is placed directly on the members (plates) during perfusion and operation of the machine. In one embodiment, the placenta, or a bag containing the placenta, is secured on the members of the device described above. The rotation of the platform, e.g., the first and the second members, provides momentum to drive the folding of placenta. In a specific embodiment, the placenta is admitted into a sterile bag, and a first portion of the placenta is placed on a first member of the platform and a second portion of the placenta is placed on a second member of the placenta. The placenta is folded while the first member and the second member are rotated relative to each other.

In various embodiments, during mechanical folding, the first member is brought to an angle of between 0° and 180°, between 45° and 135°, between 55° and 125°, or between 75° and 105° relative to said second member. In a specific embodiment, the first member is folded to an angle of about 90° relative to said second member. In certain other embodiments, the first member is folded to an angle of about 10, 20, 30, 40, 50, 60, 70, 80, 90, 100, 110, 120, 130, 140, 150, 160, 170 or 180 degrees relative to said second member.

### 5.5 Placental Cells

The methods of perfusion provided herein isolate populations of placental cells (PDPCs or HPDPCs) and perfusate (PP or HPP) that are useful for therapy in and of themselves. As used herein, the term “PP” indicates placental perfusate comprising placental cells, as obtained from a mammalian placenta; “HPP” indicates human placental perfusate comprising placental cells, as obtained from a human placenta; the term “PDPC” means mammalian placenta derived perfusate cells, which are perfusate-collected cells that have been isolated from the perfusion solution; and “HPDPC” means human placenta derived perfusate cells, which are perfusate-collected cells that have been isolated from the perfusion solution. HPP and HPDPC are described in U.S. Pat. No. 7,638,141 “Human Placental Perfusate and Placental Cells Isolated Therefrom,” the disclosure of which is hereby incorporated by reference in its entirety. Therapeutic uses of HPP and HPDPC are described, e.g., in U.S. Patent Application Publication No. 2009/0252710, filed Sep. 29, 2008, entitled “Tumor Suppression Using Human Placental Perfusate and Human Placenta Derived Intermediate Natural Killer Cells,” the disclosure of which is hereby incorporated by reference in its entirety. HPP and HPDPC can also be used as a source of pluripotent or multipotent placental cells, e.g., CD34⁺ placental cells (e.g, placental hematopoietic cells) or tissue culture plastic-adherent CD34⁻ multipotent placental cells. Such tissue culture plastic-adherent CD34 multipotent placental cells are described in detail in, e.g., U.S. Pat. No. 7,468,276 and in U.S. Patent Application Publication No. 2007/0275362, the disclosures of each of which are hereby incorporated by reference in its entirety.

Cells collected from the placenta by perfusion can be isolated or separated from perfusate by any method known in the art to separate cells from a solution, e.g., centrifugation, antibody separation, magnetic separation, flow cytometry, or the like. Similarly, such techniques can be used to separate one subpopulation of cells from perfusate, or from total nucleated placental cells (HPDPCs), from another. In one embodiment, cells, e.g., HPDPC are separated from HPP by centrifugation, e.g., at 150×g for 15 minutes at room temperature, which separates cells from, e.g., contaminating debris and platelets. HPDPC cells may be initially, or further, separated from HPP using density gradient centrifugation, e.g., using Ficoll or Percoll. In another embodiment, for example, HPP is concentrated to about 200 ml, gently layered over Ficoll, and centrifuged at about 1100×g for 20 minutes at 22° C., and the low-density interface layer of cells is collected for further processing. Contaminating erythrocytes in HPP and/or HPDPC can be removed by known methods, e.g., use of a plasma extractor; contacting the HPP and/or HPDPC with a solution comprising an acid that causes agglutination of erythrocytes, followed by filtration (see, e.g., U.S. Pat. No. 5,118,428), or the like.

In certain embodiments, the HPP and/or HPDPCs comprise placental hematopoietic cells, e.g., CD34⁺ stem or progenitor cells, which are not obtained from umbilical cord blood. In certain embodiments, the placental hematopoietic cells may be separated or isolated from HPP, or from HPDPCs, e.g., using antibodies to CD34 by, e.g., flow cytometry, magnetic cell separation or the like.

Tissue culture plastic-adherent placental cells, e.g., placental stem cells, may be isolated from HPP or HPDPC, e.g., by culturing the HPP or HPDPC on tissue culture plastic for 24-48 hours in growth medium, e.g., MDEM, IMDM, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dexamethasone L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 1% to 20% FBS, EGF, and heparin; α-MEM (minimal essential medium) comprising 10% FBS, GLUTAMAX™ and gentamicin; DMEM comprising 10% FBS, GLUTAMAX™ and gentamicin or the like, followed by removal of non-adherent cells, growth of the remaining cells to confluence, and passaging of the adherent cells in the same or similar medium. Methods of isolating or separating the tissue culture plastic-adherent placental cells from HPP and/or HPDPC are found, e.g., in U.S. Pat. No. 7,468,276, and in U.S. Patent Application Publication No. 2007/0275362, the disclosures of each of which are incorporated herein by reference in their entireties.

In certain embodiments, the HPP and/or HPDPCs comprise placental cells that are tissue culture plastic adherent and CD34⁻, CD10⁺ and CD105⁺ as detected by flow cytometry. In another specific embodiment, the CD34⁻, CD10⁺, CD105⁺ placental cells have the potential to differentiate into cells of a neural phenotype, cells of an osteogenic phenotype, and/or cells of a chondrogenic phenotype, e.g., either in vitro or in vivo, or both. In another specific embodiment, the CD34⁻, CD10⁺, CD105⁺ placental cells are additionally CD200⁺. In another specific embodiment, the CD34⁻, CD10⁺, CD105⁺ placental cells are additionally CD45⁻ or CD90⁺. In another specific embodiment, the CD34⁻, CD10⁺, CD105⁺ placental cells are additionally CD45⁻ and CD90⁺, as detected by flow cytometry. In another specific embodiment, the CD34⁻, CD10⁺, CD105⁺, CD200⁺ placental cells are additionally CD90⁺ or CD45⁻, as detected by flow cytometry. In a specific embodiment, the CD34⁻, CD10⁺, CD105⁺, CD200⁺ placental cells are additionally one or more of CD44⁺, CD45⁻, CD90⁺, CD166⁺, KDR⁺, or CD133⁻. In another specific embodiment, the CD34⁻, CD10⁺, CD105⁺, CD200⁺ placental cells are additionally CD44⁺, CD45⁻, CD90⁺, CD166⁺, KDR⁺, and CD133⁻.

In another specific embodiment, the CD34⁻, CD10⁺, CD105⁺, CD200⁺ placental cells are additionally CD90⁺and CD45⁻, as detected by flow cytometry, i.e., the placental cells are CD34⁻, CD10⁺, CD45⁻, CD90⁺, CD105⁺ and CD200⁺. In another specific embodiment, said CD34⁻, CD10⁺, CD45⁻, CD90⁺, CD105⁺, CD200⁺ placental cells are additionally CD44⁺, CD80⁻ and/or CD86⁻. In another specific embodiment, said CD34⁻, CD10⁺, CD44⁺, CD45⁻, CD90⁺, CD105⁺, CD200⁺ placental cells are additionally one or more of CD80⁻, CD86⁻, CD117⁻, CD133⁻, cytokeratin⁺, KDR⁺, HLA-A,B,C⁺, HLA-DR,DP,DQ⁻, and HLA-G⁻. In another specific embodiment, the CD34⁻, CD10⁺, CD105⁺ placental cells are additionally one or more of SSEA1⁻, SSEA3⁻and/or SSEA4⁻. In another specific embodiment, the CD34⁻, CD10⁺, CD105⁺ placental cells are additionally SSEA1, SSEA3 and SSEA4.

In another embodiment, said placental cells or population of isolated placental cells are CD34⁻, CD10⁺, CD105⁺ and CD200⁺, and one or more of CD44⁺, CD45⁻, CD90⁺, CD166⁺, KDR, or CD133. In a more specific embodiment, said placental cells or population of such isolated placental cells are CD34⁻, CD10⁺, CD105⁺ and CD200⁺, CD44⁺, CD45⁻, CD90⁺, CD166⁺, KDR⁻, and CD133⁻. In another embodiment, said placental cells or population of such isolated placental cells are CD34, CD10⁺, CD105⁺ and CD200⁺, and one or more of HLA ABC⁺, HLA DR,DQ,DP⁻, CD80⁻, CD86⁻, CD98⁻, or PD-L1. In a more specific embodiment, said placental cells or population of such isolated placental cells are CD34⁻, CD10⁺, CD105⁺ and CD200⁺, HLA ABC⁺, HLA DR,DQ,DP⁻, CD80⁻, CD86⁻, CD98⁻, and PD-L1. In certain embodiments, said placental cells or population of such isolated placental cells are CD34⁻, CD10⁺, CD105⁺ and CD200⁺, and one or more of CD3⁻, CD9⁻, CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ cells or populations of such isolated placental cells described above are additionally one or more of CD29⁺, CD38⁻, CD44⁺, CD54⁺, SH3⁺ or SH4⁺. In another specific embodiment, the placental cells or population of isolated placental cells are additionally CD44⁺. In another specific embodiment of any of the isolated CD34⁻, CD10⁺, CD105⁺ placental cells or population of such isolated placental cells above, the cells or population of cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34⁻, CD10⁺, CD105⁺ placental cells are additionally one or more of CD3⁻, CD9⁻, CD13⁺, CD29⁺, CD33⁺, CD38⁻, CD44 44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD146⁺, CD166⁺, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another embodiment, the CD34⁻, CD10⁺, CD105⁺ placental cells are additionally CD3⁻, CD9⁻, CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD146⁺, CD166⁺, CD184/CXCR4⁻, CD200⁺, CD133⁻, OCT-4⁺, SSEA3⁻, SSEA4⁻, ABC-p⁺, KDR⁻(VEGFR2⁻), HLA-A,B,C⁺, HLA-DP,DQ,DR⁻, HLA-G⁻, and Programmed Death-1 Ligand (PDL1)⁺.

In another specific embodiment, any of the placental cells described herein, contained in HPP or HPDPC are ABC-p⁺, as detected by flow cytometry, or OCT-4⁺ (POU5F1⁺), as determined by RT-PCR, wherein ABC-p is a placenta-specific ABC transporter protein (also known as breast cancer resistance protein (BCRP) and as mitoxantrone resistance protein (MXR)), and OCT-4 is the Octamer-4 protein (POU5F1). In another specific embodiment, any of the placental cells described herein are additionally SSEA3⁻ or SSEA4⁻, as determined by flow cytometry, wherein SSEA3 is Stage Specific Embryonic Antigen 3, and SSEA4 is Stage Specific Embryonic Antigen 4. In another specific embodiment, any of the placental cells described herein are additionally SSEA3⁻ and SSEA4⁻.

In another specific embodiment, any of the placental cells described above, contained within HPP or HPDPC, are one or more of MHC-I⁺ (e.g., HLA-A,B,C⁺), MHC-II⁻ (e.g., HLA-DP,DQ,DR⁻) or HLA-G⁻. In another specific embodiment, any of the placental cells described herein are one or more of MHC-I⁺ (e.g., HLA-A,B,C⁺), MHC-II⁻(e.g., HLA-DP,DQ,DR⁻) and HLA-G⁻.

In certain embodiments, the tissue culture plastic adherent placental cells, contained within HPP or HPDPC, are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3³¹, SSEA4⁻, OCT-4⁺, and ABC-p⁺. In a specific embodiment, the placental cells are OCT-4⁺ and ABC-p⁺. In another specific embodiment, the isolated placental cells, contained within HPP or HPDPC, are OCT-4⁺ and CD34⁻, wherein said placental cells have at least one of the following characteristics: CD10⁺, CD29⁺, CD44⁺, CD45⁻, CD54⁺, CD90⁺, SH3⁺, SH4⁺, SSEA3⁻, and SSEA4⁻. In another specific embodiment, the isolated placental cells are OCT-4⁺, CD34⁻, CD10⁺, CD29⁺, CD44⁺, CD45⁻, CD54⁺, CD90⁺, SH3⁺, SH4⁺, SSEA3⁻, and SSEA4⁻. In another embodiment, the placental cells are OCT-4⁺, CD34⁻, SSEA3⁻, and SSEA4⁻. In another specific embodiment, the placental cells are OCT-4⁺and CD34⁻, and is either SH2⁺ or SH3⁺. In another specific embodiment, the placental cells are OCT-4⁺, CD34⁻, SH2⁺, and SH3⁺. In another specific embodiment, the placental cells are OCT-4⁺, CD34⁻, SSEA3⁻, and SSEA4⁻, and are either SH2⁺ or SH3⁺. In another specific embodiment, the placental cells are OCT-4⁺ and CD34⁻, and either SH2⁺or SH3⁺, and is at least one of CD10⁺, CD29⁺, CD44⁺, CD45⁻, CD54⁺, CD90⁺, SSEA3⁻, or SSEA4⁻. In another specific embodiment, the placental cells are OCT-4⁺, CD34⁻, CD10⁺, CD29⁺, CD44⁺, CD45⁻, CD54⁺, CD90 ⁺, SSEA3⁻, and SSEA4⁻, and either SH2⁺ or SH3⁺.

In another embodiment, the tissue culture plastic adherent placental cells, contained within HPP or HPDPC, are SH2⁺, SH3⁺, SH4⁺ and OCT-4⁺. In another specific embodiment, the placental cells, contained within HPP or HPDPC, are CD10⁺, CD29⁺, CD44⁺, CD54⁺, CD90⁺, CD34⁻, CD45⁻, SSEA3⁻, and/or SSEA4⁻. In another embodiment, the placental cells, contained within HPP or HPDPC, are SH2⁺, SH3⁺, SH4⁺, SSEA3⁻ and SSEA4⁻. In another specific embodiment, the placental cells, contained within HPP or HPDPC, are SH2⁺, SH3⁺, SH4⁺, SSEA3⁻ and SSEA4⁻, CD10⁺, CD29⁺, CD44⁺, CD54⁺, CD90⁺, OCT-4⁺, CD34⁻ and/or CD45⁻.

In another embodiment, the placental cells, contained within HPP or HPDPC, are CD10⁺, CD29^{+,} CD34⁻, CD44^{+,} CD45⁻, CD54⁺, CD90⁺, SH2⁺, SH3⁺, and SH4⁺; wherein said isolated placental cells are additionally one or more of OCT-4⁺, SSEA3⁻ or SSEA4⁻.

In certain embodiments, placental cells, contained within HPP or HPDPC, are CD200⁺ or HLA-G⁻. In a specific embodiment, the placental cells are CD200⁺ and HLA-G⁻. In another specific embodiment, the placental cells are additionally CD73⁺ and CD105⁺. In another specific embodiment, the placental cells are additionally CD34⁻, CD38⁻ or CD45⁻. In another specific embodiment, the placental cells are additionally CD34⁻, CD38⁻ and CD45⁻. In another specific embodiment, said placental cells are CD34⁻, CD38⁻, CD45⁻, CD73⁺ and CD105⁺. In another specific embodiment, said CD200⁺ or HLA-G⁻ placental cells facilitate the formation of embryoid-like bodies in a population of placental cells comprising the isolated placental cells, under conditions that allow the formation of embryoid-like bodies.

In another embodiment, the tissue culture plastic adherent placental cells, contained within HPP or HPDPC, are CD73⁺, CD105⁺, and CD200⁺. In another specific embodiment, the placental cells are additionally HLA-G⁻. In another specific embodiment, the placental cells are additionally CD34⁻, CD38⁻ or CD45⁻. In another specific embodiment, the placental cells are additionally CD34⁻, CD38⁻ and CD45⁻. In another specific embodiment, the placental cells are additionally CD34⁻, CD38⁻, CD45⁻, and HLA-G⁻. In another specific embodiment, the CD73⁺, CD105⁺, and CD200⁺ placental cells facilitate the formation of one or more embryoid-like bodies in a population of placental cells comprising the isolated placental cells, when the population is cultured under conditions that allow the formation of embryoid-like bodies.

In certain other embodiments, the tissue culture plastic-adherent placental cells, contained within HPP or HPDPC, are one or more of CD10⁺, CD29⁺, CD34⁻, CD38⁻, CD44⁺, CD45⁻, CD54⁺, CD90⁺, SH2⁺, SH3⁺, SH4⁺, SSEA3−, SSEA4⁻, OCT-4⁺, HLA-G⁻ or ABC-p⁺. In a specific embodiment, the placental cells are CD10⁺, CD29⁺, CD34⁻, CD38⁻, CD44⁺, CD45⁻, CD54⁺, CD90⁺, SH2⁺, SH3⁺, SH4⁺, SSEA3−, SSEA4⁻, and OCT-4⁺. In another specific embodiment, the placental cells are CD10⁺, CD29⁺, CD34⁻, CD38⁻, CD45⁻, CD54⁺, SH2⁺, SH3⁺, and SH4⁺. In another specific embodiment, the placental cells are CD10⁺, CD29⁺, CD34⁻, CD38⁻, CD45⁻, CD54⁺, SH2⁺, SH3⁺, SH4⁺ and OCT-4⁺. In another specific embodiment, the placental cells are CD10⁺, CD29⁺, CD34⁻, CD38⁻, CD44⁺, CD45⁻, CD54⁺, CD90⁺, HLA-G, SH2⁺, SH3⁺, SH4⁺. In another specific embodiment, the placental cells are OCT-4⁺ and ABC-p⁺. In another specific embodiment, the placental cells are SH2⁺, SH3⁺, SH4⁺ and OCT-4⁺. In another embodiment, the placental cells are OCT-4⁺, CD34⁻, SSEA3⁻, and SSEA4⁻. In a specific embodiment, said OCT-4⁺, CD34, SSEA3, and SSEA4 placental cells are additionally CD10⁺, CD29⁺, CD34⁻, CD44⁺, CD45⁻, CD54⁺, CD90⁺, SH2⁺, SH3⁺, and SH4⁺. In another embodiment, the placental cells are OCT-4⁺ and CD34⁻, and either SH3⁺ or SH4⁺. In another embodiment, the placental cells are CD34⁻ and one or more of CD10⁺, CD29⁺, CD44⁺, CD54⁺, CD90⁺, or OCT-4⁺.

In another embodiment, the tissue culture plastic adherent placental cells, contained within HPP or HPDPC, are CD200⁺ and OCT-4⁺. In a specific embodiment, the placental cells CD73⁺ and CD105⁺. In another specific embodiment, said placental cells are HLA-G⁻. In another specific embodiment, said CD200⁺, OCT-4⁺ placental cells are CD34⁻, CD38⁻ or CD45⁻. In another specific embodiment, said CD200⁺, OCT-4⁺ placental cells placental cells are CD34⁻, CD38⁻ and CD45⁻. In another specific embodiment, said CD200⁺, OCT-4⁺ placental cells are CD34⁻, CD38⁻, CD45⁻, CD73⁺, CD105⁺ and HLA-G⁻. In another specific embodiment, the CD200⁺, OCT-4⁺ placental cells facilitate the production of one or more embryoid-like bodies by a population of placental cells that comprises the isolated cells, when the cells are cultured under conditions that allow the formation of embryoid-like bodies.

In another embodiment, the tissue culture plastic-adherent placental cells, contained within HPP or HPDPC, are CD73⁺, CD105⁺ and HLA-G⁻. In another specific embodiment, the CD73⁺, CD105⁺ and HLA-G⁻ placental cells are additionally CD34⁻, CD38⁻ or CD45⁻. In another specific embodiment, the CD73⁺, CD105⁺, HLA-G⁻ placental cells are additionally CD34⁻, CD38⁻ and CD45⁻. In another specific embodiment, the CD73⁺, CD105⁺, HLA-G⁻ placental cells are additionally OCT-4⁺. In another specific embodiment, the CD73⁺, CD105⁺, HLA-G⁻ placental cells are additionally CD200⁺. In another specific embodiment, the CD73⁺, CD105⁺, HLA-G⁻ placental cells are additionally CD34⁻, CD38⁻, CD45⁻, OCT-4⁺ and CD200⁺. In another specific embodiment, the CD73⁺, CD105⁺, HLA-G⁻ placental cells facilitate the formation of embryoid-like bodies in a population of placental cells comprising said cells, when the population is cultured under conditions that allow the formation of embryoid-like bodies.

In another embodiment, the tissue culture plastic adherent placental cells, contained within HPP or HPDPC, are CD73⁺ and CD105⁺ and facilitate the formation of one or more embryoid-like bodies in a population of isolated placental cells comprising said CD73⁺, CD105⁺ cells when said population is cultured under conditions that allow formation of embryoid-like bodies. In another specific embodiment, said CD73⁺, CD105⁺ placental cells are additionally CD34⁻, CD38⁻ or CD45⁻. In another specific embodiment, said CD73⁺, CD105⁺ placental cells are additionally CD34, CD38 and CD45. In another specific embodiment, said CD73⁺, CD105⁺ placental cells are additionally OCT-4⁺. In another specific embodiment, said CD73⁺, CD105⁺ placental cells are additionally OCT-4⁺, CD34⁻, CD38⁻and CD45⁻.

In another embodiment, the tissue culture plastic adherent placental cells, contained within HPP or HPDPC, are OCT-4⁺ and facilitate formation of one or more embryoid-like bodies in a population of isolated placental cells comprising said cells when cultured under conditions that allow formation of embryoid-like bodies. In a specific embodiment, said OCT-4⁺ placental cells are additionally CD73⁺ and CD105⁺. In another specific embodiment, said OCT-4⁺ placental cells are additionally CD34⁻, CD38⁻, or CD45⁻. In another specific embodiment, said OCT-4⁺ placental cells are additionally CD200⁺. In another specific embodiment, said OCT-4⁺ placental cells are additionally CD73⁺, CD105⁺, CD200⁺, CD34⁻, CD38⁻, and CD45⁻.

In another embodiment, the tissue culture plastic adherent placental cells, contained within HPP or HPDPC, are HLA-A,B,C⁺, CD45⁻, CD133⁻ and CD34⁻ placental cells. In another embodiment, the tissue culture plastic adherent placental cells, contained within HPP or HPDPC, are CD10⁺, CD13⁺, CD33⁺, CD45⁻, CD117⁻ and CD133⁻ placental cells. In another embodiment, the tissue culture plastic adherent placental cells, contained within HPP or HPDPC, are CD10⁻, CD33⁻, CD44⁺, CD45⁻, and CD117⁻ placental cells. In another embodiment, the tissue culture plastic adherent placental cells, contained within HPP or HPDPC, are CD10⁻, CD13⁻, CD33⁻, CD45⁻, and CD117⁻ placental cells. In another embodiment, the tissue culture plastic adherent placental cells, contained within HPP or HPDPC, are HLA A,B,C⁺, CD45⁻, CD34⁻, and CD133⁻, and are additionally CD10⁺, CD13⁺, CD38⁺, CD44⁺, CD90⁺, CD105⁺, CD200⁺ and/or HLA-G⁻, and/or negative for CD117.

In another embodiment, the tissue culture plastic adherent placental cells, contained within HPP or HPDPC, are CD200⁺ and CD10⁺, as determined by antibody binding, and CD117⁻, as determined by both antibody binding and RT-PCR. In another embodiment, the tissue culture plastic adherent placental cells, contained within HPP or HPDPC, are CD10⁺, CD29, CD54⁺, CD200⁺, HLA-G, MHC class I⁺ and β-2-microglobulin⁺.

In another embodiment, the tissue culture plastic adherent placental cells, contained within HPP or HPDPC, are one or more of CD10⁺, CD29⁺, CD44⁺, CD45⁻, CD54/ICAM⁺, CD62E, CD62L, CD62P, CD80, CD86, CD103, CD104, CD105⁺, CD106/VCAM⁺, CD144/VE-cadherin^{low}, CD184/CXCR4⁻, β2-microglobulin^{low}, MHC-II⁻, HLA-G^{low}, and/or PDL1^{low}. In a specific embodiment, the placental cells are at least CD29⁺ and CD54⁺. In another specific embodiment, the placental cells are at least CD44⁺ and CD106⁺. In another specific embodiment, the placental cells are at least CD29⁺.

In another embodiment, the tissue culture plastic adherent placental cells, contained within HPP or HPDPC, are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, where ABC-p is a placenta-specific ABC transporter protein (also known as breast cancer resistance protein (BCRP) and as mitoxantrone resistance protein (MXR)).

In any of the embodiments of HPP, HPDPC and/or tissue culture plastic adherent placental cells contained within HPP or HPDPC, the cells are a mixture of maternal and fetal (non-maternal) cells, e.g., at least 20%, 30%, 40%, 50%, 60%, 70% or 80% maternal cells. In certain other embodiments of HPP, HPDPC and/or tissue culture plastic adherent placental cells contained within HPP or HPDPC, the cells are substantially only fetal cells, e.g., at least 85%, 90%, 95%, 98%, or 99% fetal cells.

### 6. Example

### 6.1 Example 1

### Collection of Placental Stem Cells by Closed-Circuit Perfusiion in a Sterile Bag

A post-partum placenta is obtained within 24 hours after birth. The amniotic membrane and chorion are separated and processed. Starting from the edge of the placental membrane, the amniotic membrane is separated from the chorion using blunt dissection with the fingers. When the amniotic membrane is entirely separated from the chorion, the amniotic membrane is cut around the base of the umbilical cord with scissors, and detached from the placental disk. The amniotic membrane can be discarded, or processed, e.g., to obtain stem cells by enzymatic digestion, or to produce, e.g., an amniotic membrane biomaterial.

The fetal side of the remaining placental material is cleaned of all visible blood clots and residual blood using sterile gauze, and is then sterilized by wiping with an iodine swab than with an alcohol swab. The umbilical cord is then clamped crosswise with a sterile hemostat beneath the umbilical cord clamp, and the hemostat is rotated away, pulling the cord over the clamp to create a fold. The cord is then partially cut below the hemostat to expose a cross-section of the cord supported by the clamp. Alternatively, the cord is clamped with a sterile hemostat. The cord is then placed on sterile gauze and held with the hemostat to provide tension. The cord is then cut straight across directly below the hemostat, and the edge of the cord near the vessel is re-clamped.

The vessels exposed as described above, usually a vein and two arteries, are identified, and opened as follows. A closed alligator clamp is advanced through the cut end of each vessel, taking care not to puncture the clamp through the vessel wall. Insertion is halted when the tip of the clamp is slightly above the base of the umbilical cord. The clamp is then slightly opened, and slowly withdrawn from the vessel to dilate the vessel.

First plastic tubing suitable for perfusion is passed through a port in a sterile bag, which is suitable for containing the placenta during perfusion, and pulled through the opening in the bag. This end of the tubing is inserted into each of the placental arteries; the other end is connected to a peristaltic pump that passes perfusion solution to a collection bag. Second plastic tubing, connected to a 250 mL collection bag via a coupler, is also passed through the port and out the opening of the bag, and is inserted into the placental vein. The tubing is taped into place on the umbilical cord. The placenta is then placed into the sterile bag under aseptic conditions, and the slack tubing is either drawn through the port or allowed to coil inside the bag, and the opening of the bag is sealed.

A small volume of sterile injection grade 0.9% NaC1 solution is used to check for leaks. If no leaks are present, the pump speed is increased, and about 750 mL of the injection grade 0.9% NaCl solution is pumped through the placental vasculature. Perfusion can be aided by gently massaging the placental disk from the outer edges to the cord. When a collection bag is full, the bag is removed from the coupler connecting the tubing to the bag, and a new bag is connected to the tube.

When collection is finished, the collection bags are weighed and balanced for centrifugation. After centrifugation, each bag is placed inside a plasma extractor without disturbing the pellet of cells. The supernatant within the bags is then removed and discarded. The bag is then gently massaged to resuspend the cells in the remaining supernatant. Using a sterile 1 mL syringe, about 300-500 μL of cells is withdrawn from the collection bag, via a sampling site coupler, and transferred to a 1.5 mL centrifuge tube. The weight and volume of the remaining perfusate are determined, and ⅓ volume of hetastarch is added to the perfusate and mixed thoroughly. The number of cells per mL is determined. Red blood cells are removed from the perfusate using a plasma extractor.

After perfusion and collection of cells is complete, the bag, tubing and placenta are discarded.

### 6.2 Example 2

### Collection of Placental Stem Cells by Physical Manipulation in a Sterile Bag

A post-partum placenta is obtained within 24 hours after birth. The amniotic membrane and chorion are separated and processed. Starting from the edge of the placental membrane, the amniotic membrane is separated from the chorion using blunt dissection with the fingers. When the amniotic membrane is entirely separated from the chorion, the amniotic membrane is cut around the base of the umbilical cord with scissors, and detached from the placental disk. The amniotic membrane can be discarded, or processed, e.g., to obtain stem cells by enzymatic digestion, or to produce, e.g., an amniotic membrane biomaterial.

The fetal side of the remaining placental material is cleaned of all visible blood clots and residual blood using sterile gauze, and is then sterilized by wiping with an iodine swab than with an alcohol swab. The umbilical cord is then clamped crosswise with a sterile hemostat beneath the umbilical cord clamp, and the hemostat is rotated away, pulling the cord over the clamp to create a fold. The cord is then partially cut below the hemostat to expose a cross-section of the cord supported by the clamp. Alternatively, the cord is clamped with a sterile hemostat. The cord is then placed on sterile gauze and held with the hemostat to provide tension. The cord is then cut straight across directly below the hemostat, and the edge of the cord near the vessel is re-clamped.

The vessels exposed as described above, usually a vein and two arteries, are identified, and opened as follows. A closed alligator clamp is advanced through the cut end of each vessel, taking care not to puncture the clamp through the vessel wall. Insertion is halted when the tip of the clamp is slightly above the base of the umbilical cord. The clamp is then slightly opened, and slowly withdrawn from the vessel to dilate the vessel.

First plastic tubing suitable for perfusion is passed through a port in a sterile bag, which is suitable for containing the placenta during perfusion, and pulled through the opening in the bag. This end of the tubing is inserted into each of the placental arteries; the other end is connected to a peristaltic pump. Second plastic tubing, connected to a collection bag, is also passed through the port and out the opening of the bag, and is inserted into the placental vein. The tubing is taped into place on the umbilical cord. The placenta is then placed into the sterile bag under aseptic conditions. The slack tubing is either drawn through the port or allowed to coil inside the bag, and the opening of the bag is sealed.

The sterile bag containing the placental sample is then secured on the platform of a device as shown in FIG. 1 and FIG. 2. The placenta is perfused by closed circuit perfusion as described in Section 6.1, above. During perfusion, the placenta is folded by the device approximately 15-20 times per minute for the duration of perfusion, for a total of about 110 to 150 foldings. The placenta is folded between approximately 0° and 90° each time. The perfusate is collected and the red blood cells are removed from the perfusate as in Section 6.1, above.

Compared to a standard method of perfusing, which includes manual manipulation of the placenta during perfusion by massaging the placental vasculature with the fingertips, folding of the placenta results in a detectably higher number of nucleated placental cells recovered per 100 mL perfusion solution.

After perfusion and collection of cells is complete, the bag, tubing and placenta are removed from the device and discarded.

### Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references, and all patents and patent applications, cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of collecting placental cells from a mammalian placenta, comprising perfusing said mammalian placenta with a perfusion solution in an amount and for a time sufficient to collect a detectable amount of said placental cells, wherein said placenta is contained within a sterile bag during said perfusing.

2. A method of collecting placental cells from a mammalian placenta, comprising perfusing said mammalian placenta with a perfusion solution in an amount and for a time sufficient to collect a detectable amount of said placental cells, wherein a first portion of said placenta is folded at least once towards a second portion of said placenta during said perfusing, and wherein said first portion of said placenta is different from said second portion of said placenta.

3. The method of claim 2, wherein said placenta comprises a first plurality of portions and a second plurality of portions, and wherein at least one of said first plurality of portions is folded towards one of said second plurality of portions during said perfusing.

4. The method of claim 3, wherein each of said first plurality of portions of said placenta is different from each of said second plurality of portions of said placenta.

5. The method of claim 2, wherein said first portion of said placenta is disposed at an angle ranging from about 45° to about 135° relative to said second portion while said placenta is folded.

6. The method of claim 2, wherein said first portion of said placenta is disposed at an angle ranging from about 75° to about 105° relative to said second portion while said placenta is folded.

7. The method of claim 2, wherein said placenta is folded a plurality of times during said perfusing.

8. The method of claim 2, wherein said mammalian placenta is contained within a sterile bag while said placenta is folded.

9. The method of claim 2, wherein said placenta is folded manually.

10. The method of claim 8, wherein said placenta is folded mechanically.

11. The method of claim 10, wherein said placenta is folded by securing said bag on a platform, wherein said platform comprises a first member and a second member, and wherein said first member and second member are connected and rotatable such that said first member is aligned at an angle ranging from about 0° to about 180° to said second member.

12. The method of claim 11, wherein said first portion of said placenta is placed on said first member and said second portion of said placenta is placed on said second member of said placenta, and wherein said placenta is folded while said first member and second member are rotated.

13. The method of claim 11, wherein said first member is aligned at an angle ranging from about 45° to about 135° to said second member.

14. The method of claim 11, wherein said first member is aligned at an angle ranging from about 75° to about 105° to said second member.

15. The method of claim 2, wherein said perfusing is performed using a first volume of between about 100 ml and about 1000 ml of said perfusion solution.

16. The method of claim 2, further comprising continuing said perfusing using a second volume of about 100 ml to about 1000 ml of said perfusion solution, said second volume being collected separately from said first volume.

17. The method of claim 2, wherein said perfusion solution comprises heparin, ethylene diamine tetra acetic acid (EDTA) or creatine phosphate dextrose (CPDA).

18. The method of claims 1, wherein said perfusion solution comprises a growth factor or a cytokine.

19. The method of claim 2, further comprising separating said placental cells from cells other than placental cells and said perfusion solution.

20. The method of claim 2, further comprising separating said placental cells from said perfusion solution.
